(19) United States
(12) Patent Application Publication
(10) Pub. No.: US 2010/0331832 A1
(43) Pub. Date: Dec. 30, 2010

(54) METHOD FOR TREATING SKIN

(76) Inventor: BORIS VAYNBERG, Zikron Yaakov (IL)

Correspondence Address: SMITH FROHWEIN TEMPEL GREENLEE BLAHA, LLC, Two Ravinia Drive, Suite 700, ATLANTA, GA, 30346, US

(21) Appl. No.: 12/491,003

(22) Filed: Jun. 24, 2009

(51) Int. Cl. A61B 18/20

(52) U.S. Cl. 606/9

(57) ABSTRACT

Techniques for the treatment of skin segments by application of optical radiation are limited due to dark skin pigmentation. The application of a whitening substance is utilized to temporarily lighten a targeted skin segment. In the temporarily lightened state, optical radiation for skin treatment is applied in a more effective manner.

### CROSS-REFERENCE TO RELATED APPLICATIONS

The following United States patents are related to the present disclosure and are incorporated herein by reference to provide an overall understanding of the various aspects, features and embodiments of the present disclosure:

U.S. Pat. No. 7,422,734, bearing the title of METHODS OF PRODUCING LEGNIN PEROXIDASE AND ITS USE IN SKIN AND HAIR LIGHTENING and issued to Belinky et. al. on Sep. 9, 2008; and

U.S. Pat. No. 6,702,808, bearing the title of DEVICE AND METHOD FOR TREATING SKIN and issued to Kreindel on Mar. 9, 2004.

### TECHNOLOGY HELD

The apparatus and method relate to the field of cosmetic skin treatment by optical radiation.

### BACKGROUND

In course of the last two decades, a variety of optical radiation based skin treatments and hair removal procedures have been developed. These optical radiation based aesthetic technologies include, among other technologies, the application of laser, and/or intense pulsed light (IPL) to skin. In operation, optical radiation is applied in a continuous or a pulsed mode at a proper wavelength and power to produce the desired skin effect and cause minimal damage, if at all, to the surrounding skin or tissue. The optical radiation used for the skin treatment may be monochromatic radiation, such a laser light, or may contain a plurality of discrete wavelengths, or even a broad spectrum radiation.

Skin types can be classified according to their ability to tan in response to exposure to sun irradiation. Under this classification system, there are six classes. The first three classifications (classes I-III) represent different types of fair or light skin. The final three classifications (classes IV-VI) represent different types of dark or ethnic skins. It should be noted that some other, non-tan related skin type classifications also exist.

The use of optical radiation technology for skin treatment, and in particular dark or ethnic skin has some limitations. These limitations are due to the fact that the optical energy needs to penetrate through the epidermis before it can reach the tissue layers in which the desired effect is to be produced. Melanin content in the epidermis determines the color of the skin. Dark skin is rich in melanin, as opposed to fair skin types, and thus, it absorbs the larger amounts of optical radiation energy before the energy reaches the tissue layers set as a target for the desired effect

Therefore, in individuals with darker skin types, or those that simply have patches of dark skin, the increased pigmentation of the epidermis imposes challenging skin treatment issues. Overheating of the epidermis increases the risk of skin burns and other adverse effects that could be caused in darker skin by narrow band (laser) and broadband optical radiation treatments and in extreme cases, may result in skin inflammatory phenomena.

Because of the inherent differences between the dark and fair skin, users and operators of skin treatment equipment have to reduce the number of pulses applied continuously to the treated skin segment, use conservative treatment fluences, increase the number of passes, and reduce applied light energy or dosages to avoid the damage of dark skin. In some instances, different wavelengths have to be used for similar treatments of dark and fair skin. Reduction of the skin treatment energy leads to an increase in the number of treatments, poorer treatment results and sometimes renders the treatment almost ineffective.

Almost all types of skin develop uneven pigmented skin spots or patches. Therefore, there is a need for a safe and effective skin whitening treatment and treatment of pigmented skin patches.

### BRIEF SUMMARY

During a procedure or treatment to provide skin whitening, to reduce the damage to the skin, the segment of the skin under treatment is pre-treated by a skin bleaching or whitening substance. The application of the substance effectively reduces the level of melanin in the skin. The reduced melanin level allows for or enables an increase in the dosage of optical energy to a level that will result in the desired treatment effect while at the same time, reducing the risk of epidermal damage, shortening the treatment time, and reducing the number of treatments. Use of this method is also applicable to other skin treatments, such as wrinkles removal, collagen shrinking and any other optical radiation based cosmetic skin procedures where the treatment “target” is below the epidermis layer and where the target absorbs optical radiation in a spectral region similar to the epidermis absorption spectrum.

### BRIEF DESCRIPTION OF DRAWINGS

FIG. 1A is a schematic illustration of an exemplary skin cross section with optical radiation being applied;

FIG. 1B schematically illustrates the optical radiation intensity transmitted by a non-treated segment of the skin;

FIG. 2A is a schematic illustration of a skin cross section with optical radiation being applied after application of a skin whitening or bleaching substance;

FIG. 2B schematically illustrates the optical radiation intensity transmitted by a whitened segment of the skin;

FIGS. 3A and 3B are schematic illustrations of a pigmented skin treatment according to the present treatment method.

FIG. 4 is a flow diagram illustrating an embodiment of a skin treatment procedure incorporating the whitening method.

### DETAILED DESCRIPTION OF EXEMPLARY EMBODIMENTS

The principles and execution of the method may be understood with reference to the drawings and the accompanying description of the non-limiting, exemplary embodiments, shown in the Figures.

Uneven skin tone, age spots, freckles, dark underarms, melasma, dark acne scars, dark knees, elbows or inner thighs have been a common problem for individuals. Skin lighteners have become popular, regardless of skin color, as a solution to correct these pigmentation problems and subsequently increased one's self-esteem. These treatments however, only temporarily alleviate the problem. Optical radiation based skin treatment may allow permanent removal of the listed skin deficiencies.

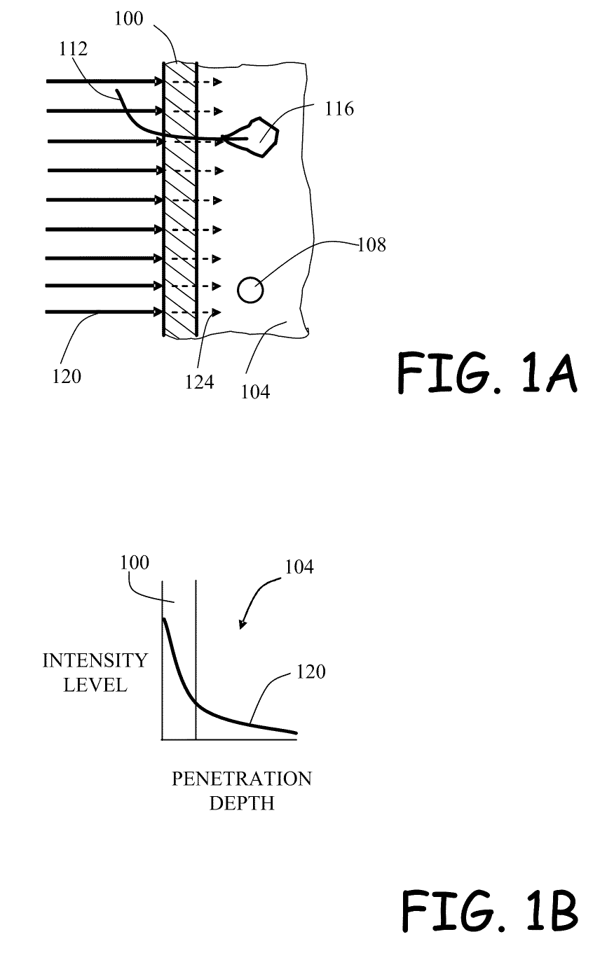

Reference is made to FIG. 1A is a schematic illustration of an exemplary skin cross section with optical radiation being applied. In the illustration, the skin cross section shows an epidermis layer 100, a dermis layer 104, a blood vessel 108, and a hair follicle 112 with its root 116. Incident on the skin optical radiation shown by arrows 120 is significantly absorbed by the melanin content contained in the epidermis layer 100. A relatively small percentage of the radiation intensity 120, or at least a significantly reduced amount of the radiation intensity 120, as shown in FIG. 1B by phantom lines 124 reaches for example, the blood vessel 108 to be treated or the hair follicle and root 116.

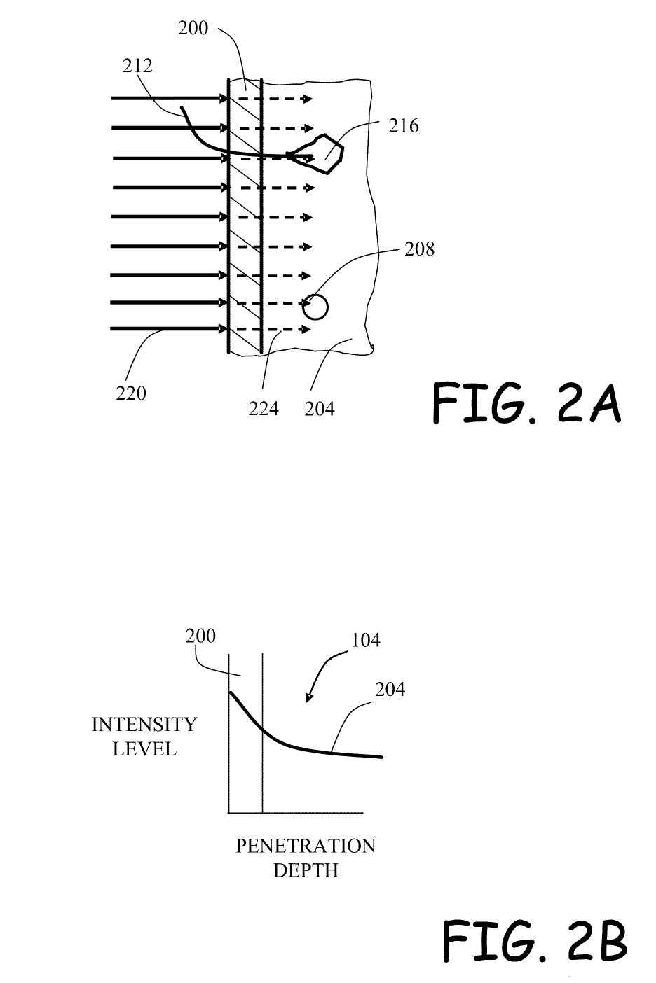

FIG. 2A is a schematic illustration of a skin cross section with optical radiation being applied after application of a skin whitening or bleaching substance. In this illustration, the skin cross section shows an epidermis layer 200, a dermis layer 204, a blood vessel 208, and a hair follicle 212 with its root 216. The skin whitening or bleaching substance may take on a variety of forms and strengths. As non-limiting examples, the substance may take the form of a cream, gel or lotion or other viscous liquid. Furthermore, the concentration, strength or potency of the substance can vary depending on skin types, sensitivities, required/desired penetration, etc. Skin whitening or bleaching creams are topically applied to the skin to temporally lessen skin pigmentation, but do not remove completely the pigmented skin or skin patches.

As a result of applying the skin whitening substance, the amount of melanin in the epidermis layer 200 is reduced, the skin level is altered and the penetration or the transmission of the optical energy through the epidermis layer 200 is significantly increased as compared to the non-treated epidermis layer 100 (FIGS. 1A and 1B). FIG. 2B schematically illustrates the optical radiation intensity 224 reaching dermis 204, blood vessel 208, hair follicle and root 216 and other targets that should be treated.

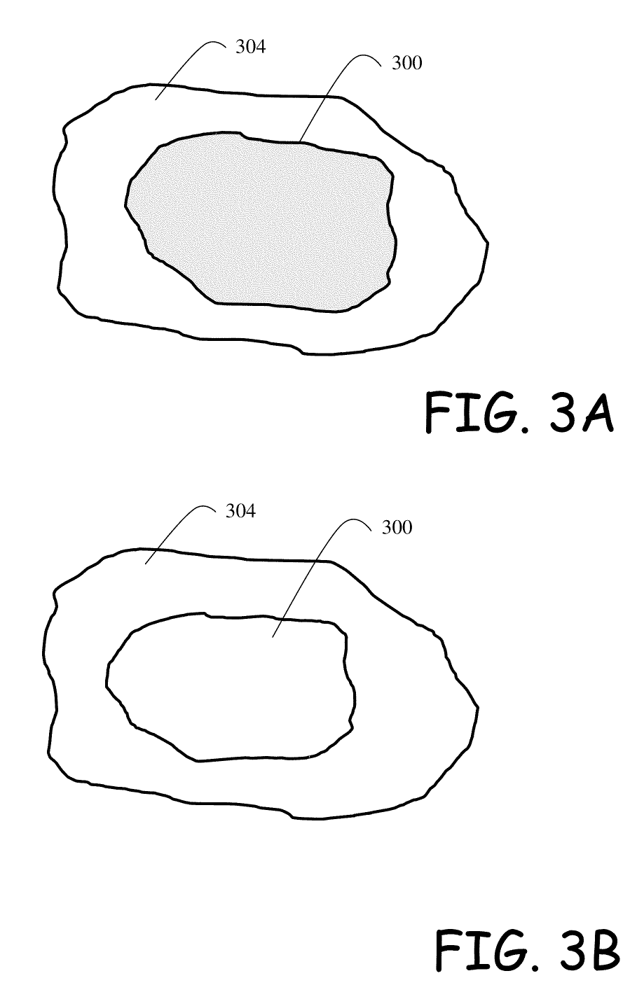

FIG. 3A is a schematic illustration of a pigmented skin patch. The illustration shows a pigmented skin patch 300 surrounded by a region of skin 304 that is non-affected by pigmentation, although the non-affected skin may be fair or dark skin.

Skin whitening or bleaching cream is topically applied to the skin to lessen the pigmented skin patches considerably, but does not remove completely the pigmented skin patches. Optical radiation treatments could most probably remove the pigmentation marks permanently, but because of the high radiation absorption by the pigmented skin, the skin temperature raises fast and the absorbed radiation may cause skin burns, without ever fully reaching the penetration conditions required for permanent pigmented skin patches removal.

FIG. 3B shows the same skin target area illustrated in FIG. 3A after the application of a skin whitening substance. The proposed method of skin treatment by optical radiation is illustrated in FIG. 3B. The method suggests bleaching or whitening a segment of the skin to be treated for example, the pigmented skin patch 300, by application of a skin whitening or bleaching substance such as cream, lotion, or gel to the targeted treatment area. The bleaching substance may be applied topically, altering the skin level and upon reaching the desired level of skin whitening (e.g., effectively changing the darker skin level to a lighter of fair skin level), the desired optical radiation may be applied to the temporarily whitened segment of the skin. The energy level or time, or what may be termed as a dosage of the optical radiation may be increased to produce a desired skin effect while mitigating or alleviating the risk of causing damage to the epidermis. The desired skin effect may be the removal of pigmented skin patches, hair removal, wrinkles removal, collagen shrinking and other cosmetic skin procedures.

Most skin whiteners currently on the market contain ingredients such as Hydroquinone (HQ), Monobenzyl ether of HQ (MBEH) Ascorbic acid, Kojic acid, Arbutin, Azealic acid, Glycyrrhetinic acid (licorice extract)) that act as direct inhibitors of tyrosinase, the enzyme in the skin pigment cells (melanocytes) that make melanin. HQ is the most widely used skin whitening agent. Despite the overall safety of the whitening agents or substances, all of them may cause potential adverse effects. For instance, in some instances, HQ may cause reversible inhibition of cellular metabolism by affecting both DNA and RNA synthesis. Further, in some instances it may cause contact dermatitis and other negative phenomena. MBEH is used in germicides, adhesive tapes, rubber, and other chemicals. The remaining substances are also not free from adverse effects. Their use in conjunction with optical radiation heating the skin and the whitening substance residing on the skin may enhance the adverse effects.

U.S. Pat. No. 7,422,734 to Belinky et al, which has been incorporated by reference, discloses a skin whitening substance including from 1 to 100 IU/gr (IU—International Unit) of lignin peroxidase enzyme application of which does not cause adverse effects.

The present method suggests using the skin whitening substance that includes lignin peroxidase enzyme for skin pre-treatment. In one embodiment the range of lignin peroxidase can be in the range of 0.005% to 0.5% and in a particular non-limiting example, may be 0.42% concentration.

The optical radiation generally is selected to match the absorption spectrum of the target skin area to be treated, for example, hair follicle, blood vessel, pigmented lesion, although radiation having a broader spectrum may be used. The optical radiation may be a narrow band radiation, such a laser or laser diode radiation or broadband radiation, generated by incandescent or Intense Pulse Light (IPL) sources.

The method disclosed may be applied to general whitening of almost all skin types. The whitening substance may be applied topically to a desired segment of skin, for example a face of a person, and upon reaching a desired dose of temporal skin whitening, optical radiation will be applied to the skin segment that has been treated by whitening substance to make the skin whitening almost permanent.

The method may be further enhanced by application of RF as disclosed in the U.S. Pat. No. 6,702,808 awarded to the same assignee as this disclosure, which supports a slower and deeper skin heating and a more consistent hair removal.

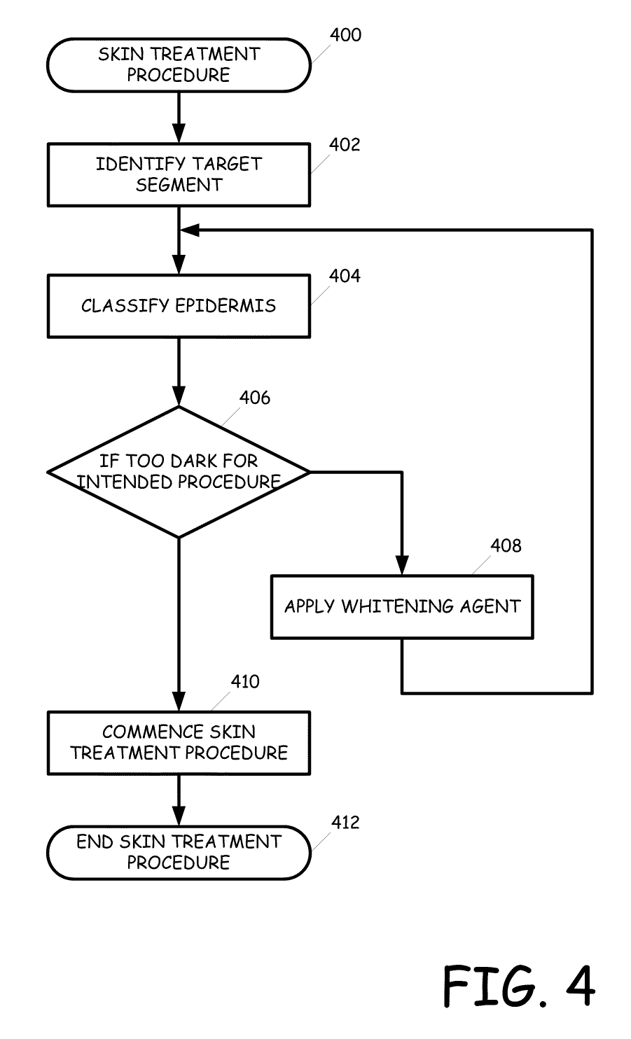

FIG. 4 is a flow diagram illustrating an embodiment of a skin treatment procedure incorporating the whitening method. The skin treatment procedure 400 commences by first conducting an examination of the target skin segment 402. Once the target skin area is identified, the epidermis or the level of pigmentation can be examined to determine the target skin segment level. The determination can be done in a variety of techniques including optical scanning, chart comparison or simply by an experienced technician. For instance, in one embodiment, the pigmentation level in the skin can be examined to determine if it is within a desired range or, if the pigmentation level is below a target pigmentation level. Once the level of the target skin segment is determined, the determination can be evaluated in accordance with the intended procedure. Some procedures may require higher optical radiation dosages than others and as such, the optimal threshold for the target skin classification may depend at least in part on the intended procedure. In any case, if the target skin segment is too dark 406 (i.e., if the pigmentation level is too high), then a whitening agent can be applied 408 to bring the pigmentation level to a required threshold level. It is also anticipated that in some circumstances, a target skin segment may be too light and as such, the disclosed method also anticipates the application of a darkening agent, such as a dye, tanning lotion, etc, in such circumstances. In various embodiments, a particular treatment may be known to produce better results when the skin pigmentation is within a specific range, above a specific threshold or below a specific threshold. Once the agent has been applied, the epidermis level can then be re-evaluated 404 to ensure that it has reached an acceptable level 406 or, that the pigmentation is within desired or required tolerances.

If the determination of the target skin segment is appropriate for the intended procedure 406, then the skin treatment procedure can be commenced 410 and then terminated after the treatment has been completed or, after a dosage has been applied 412.

In some situations, multiple treatments can be applied after one application of the whitening agent, such as multiple dosages in one setting. However, in other settings the entire procedure may require repeating if the treatment is spread out over a period of time.

The above disclosure is intended as merely exemplary, and not to limit the scope of the method, which is to be determined by reference to the appended claims.

What is claimed is:

1. A method of treating a segment of skin by optical radiation, said method comprising:
applying to the segment of skin to be treated a skin whitening substance to temporarily whiten the segment of skin; and
applying to the temporarily whitened segment of skin optical radiation.

2. The method according to claim 1, wherein the optical radiation is applied to treat the skin by providing one of a group of treatment effects consisting of: removing pigmented or vascular skin lesions, removing hair follicles, shrinking collagen, and removing wrinkles.

3. The method according to claim 1, wherein the skin whitening substance contains at least 1-100 IU/gr of lignin peroxidase enzyme.

4. The method according to claim 1, wherein the skin whitening substance is a viscous material topically applied to the skin.

5. The method according to claim 1, wherein the step of applying optical radiation further comprises the application of optical radiation using one of a group of techniques consisting of narrow band radiation and broadband radiation.

6. A method of providing treatment to a segment of skin by the application optical radiation, said method comprising:
applying a skin whitening substance to the segment of skin to be treated thereby whitening the segment of skin to a level less susceptive to optical radiation than the skin surrounding the segment of skin; and
applying optical radiation to the segment of skin.

7. The method according to claim 6, wherein the step of applying a skin whitening substance further comprises applying a viscous material containing at least 1-100 IU/gr of lignin peroxidase enzyme.

8. The method according to claim 6, wherein the skin whitening substance is a viscous material topically applied to the skin.

9. The method according to claim 6, wherein the step of applying optical radiation further comprises the step of applying optical radiation using a technique selected from a group of techniques consisting of a narrow band radiation and broadband radiation.

10. The method according to claim 6, wherein the step of applying optical radiation is to provide a treatment selected from a group of treatments consisting of: removing pigmented or vascular skin lesions, removing skin patches, removing hair follicles, collagen shrinking, and removing wrinkles.

11. A skin whitening substance for use in a skin whitening treatment by application of optical radiation, said substance being viscous and comprising at least lignin peroxidase enzyme.

12. A method of treatment for a dark skin segment by optical radiation, said method comprising:
determining the skin segment level based on pigmentation to determine if it is below a threshold pigmentation level;
applying a skin whitening substance to the segment of skin to raise the pigmentation level above the threshold pigmentation level;
applying optical radiation to the skin segment.

13. The method according to claim 12, wherein the step of applying a skin whitening substance comprises applying topically applying a viscous material to the skin segment.

14. The method according to claim 12, wherein the step of applying optical radiation further comprises the step of applying optical radiation using a technique that is selected from a group of techniques consisting of narrow band radiation and broadband radiation.

15. The method according to claim 12, wherein the step of applying optical radiation is to provide a treatment selected from a group of treatments consisting of: skin whitening, removing pigmented or vascular lesions, removing hair follicles, removing wrinkles, and collagen shrinking.

16. A method of providing a particular treatment to a target segment of skin through the use of optical radiation, said method comprising:
identifying a target segment of skin to be treated;
determining the target segment of skin level based at least in part on the level of pigmentation present in the target segment of skin;
if the target segment of skin is not within a particular range, applying a pigment altering substance to the target skin segment;
applying optical radiation to the target skin segment.

17. The method of claim 16, wherein the level of pigmentation is too high for the particular treatment and the step of applying a pigment altering substance further comprises applying a whitening agent.

18. The method of claim 17, wherein the step of applying a whitening agent further comprises the step of applying a viscous substance that contains a measure of at least 1-100 IU/gr of peroxidase enzyme.

19. The method of claim 18, wherein after the step of applying a pigment altering substance and before applying optical radiation, further comprising the steps of:
re-evaluating the target segment of skin level based at least in part on the level of pigmentation present in the target segment of skin;
if the target segment of skin is not within the particular range, re-applying a pigment altering substance to the target skin segment; and
repeating these steps until the target segment of skin is within the particular range.

20. The method of claim 16, wherein after the step of applying a pigment altering substance and before applying optical radiation, further comprising the steps of:
re-evaluating the target segment of skin level based at least in part on the level of pigmentation present in the target segment of skin;
if the target segment of skin is not within the particular range, re-applying a pigment altering substance to the target skin segment; and
repeating these steps until the target segment of skin is within the particular range.
